(19) United States
(12) Patent Application Publication
(10) Pub. No.: US 2013/0108507 A1
(43) Pub. Date: May 2, 2013

(54) Sterilized Stethoscope Barrier Assembly

(71) Applicants: Ingeborg Reiseneder, Chicago, IL (US); Renee Grais, Lincolnwood, IL (US)

(72) Inventors: Ingeborg Reiseneder, Chicago, IL (US); Renee Grais, Lincolnwood, IL (US)

(21) Appl. No.: 13/283,706

(22) Filed: Oct. 28, 2011

(51) Int. Cl. A61L 2/16

(52) U.S. Cl. 422/28

(57) ABSTRACT

The present invention provides a sterilized barrier assembly for a stethoscope defined to have at least a stethoscope head. The barrier assembly includes two layers of material. A first layer of material has a first bottom side and an aseptic first top side. The first bottom side has an adhesive material permitting the first layer to be removably attached to the stethoscope head. The second layer of material has a second bottom side and a second top side. The second bottom side has a portion with adhesive such that the second bottom side is removably attached to the aseptic first top side, therefore the second layer of material can be removed from the first layer of material exposing the aseptic first top side to the atmosphere and for use with a patient.

### FIELD OF INVENTION

The present invention relates to reducing the risk of infection by utilizing a disposable, aseptic, stethoscope barrier.

### BACKGROUND OF THE INVENTION

Ubiquitous stethoscope use, while essential in clinical practice, is also a potential vector of infection. While gowns, gloves, head covers, foot covers, and masks are accepted as essential barriers, the only sterilizing technique for the stethoscope head (bell and diaphragm) is wiping with isopropyl alcohol. There are an increasing number of infectious agents that are readily transported from patient to patient as healthcare providers use their stethoscopes without necessarily decontaminating them. Some bacteria such as methicillin resistant Staphylococcus aureous (MRSA) and Vancomycin-Resistant Entercocci (VRE), demonstrate increasing antibiotic resistance. Infections with these and other agents can be lethal, augment morbidity or hospital stays. A disposable barrier applied to the stethoscope bell and diaphragm merits use to reduce the titer of exposure.

During use, the bell or diaphragm (stethoscope head) of the stethoscope is placed against the patient's skin, and the sounds arising within the patient's body are transmitted through the stethoscope head into a tube(s) of the stethoscope, through ear pieces into the healthcare provider's ears. Thus, stethoscopes are utilized to detect and study the sounds in the patient's body.

Despite the significant advances in medical technology, stethoscopes remain an invaluable tool. Healthcare providers such as doctors, mid-level practitioners and nurses routinely employ stethoscopes to take blood pressure and to examine the chest, abdomen, and other areas of the body. Skin normally harbors a variety of bacteria, some highly dangerous such as MRSA and VRE, potentially resulting in contamination of the stethoscope. Healthcare providers typically use stethoscopes without consistent regard for aseptic technique. For example, it is recommended, although rarely done, to wipe the diaphragm and bell with isopropyl alcohol after each patient use.

To reduce the risk of spreading contamination an inexpensive, readily available, disposable barrier merits use for each patient contact.

### SUMMARY OF THE INVENTION

In accordance with the present invention there is provided a Latex Free, stethoscope barrier (material will not interfere with sound transmission) for a head piece of the stethoscope, defined to include the bell and diaphragm. The stethoscope barrier assembly as described for stethoscope use includes two layers of material. The present invention may be applied to other medical tools and/or devices. In addition, the current size and shape may be altered in the future to be used as an aseptic barrier for other medical products or if because stethoscope head shape changes. An application of an antibacterial product may be considered for use with this or like products.

A first layer of material has a first bottom side and a first top side. The first bottom side has an adhesive material permitting the first layer to be removably attached to the stethoscope head. The second layer of material has a second bottom side and a second top side. The second bottom side has a portion with adhesive such that the second bottom side is removably attached to the first top side. Therefore, when the second layer of material is removed from the first layer of material, the first top side is exposed to the atmosphere for the first time and provides for an aseptic barrier.

In another aspect the first top side may include a disinfectant and/or antiseptic agent. In this instance, when the second layer of material is removed from the first layer of material, the disinfectant and/or antiseptic agent on the first top side is exposed to the atmosphere.

In other aspects of the invention, at least one tab can be disposed on a terminal edge defined on each first and second layers of material.

In other aspects of the invention there can be further provided a packaging medium to secure to at least a portion of the first bottom side of the first layer of material to a first portion of the packaging medium. In one dispensing variation, the packaging medium is a roll of paper having a plurality of substantially identical cover assemblies secured thereto in a spaced configuration. In a second dispensing variation, the roll of paper include perforations between two substantially identical cover assemblies. In yet a third dispensing variation, the packaging medium includes a second portion over the second top side of the second layer of material. The second portion and the first portion each have a portion edge with adhesive to permit the second and first portions to be removably secured to each other.

In further accordance with the present invention, there is provided a method of providing a aseptic stethoscope head 12 defined on a stethoscope, the method comprising the steps of: (a) providing a sterilized cover assembly; (b) applying the sterilized cover assembly to the stethoscope head 12; (c) removing a layer of material from the sterilized cover assembly exposing a layer of disinfectant and/or antiseptic agent to the atmosphere; and (d) removing a remaining layer of material after use.

In a further aspect of the method the sterilized cover assembly includes a first layer of material having a first bottom side and a first top side, the first bottom side having an adhesive material permitting the first layer to be removably attached to the stethoscope head 12, the first top side having a disinfectant and/or antiseptic agent, and a second layer of material having a second bottom side and a second top side, the second bottom side having a portion with adhesive such that the second bottom side is removably attached to the first top side.

Further features will become more fully apparent in the following description of the embodiments of this invention and from the appended claims.

### BRIEF DESCRIPTION OF THE DRAWINGS

A fuller understanding of the foregoing may be had by reference to the accompanying drawings, wherein:

FIG. 1 is a perspective view of a prior art stethoscope;

FIG. 2A is a top view of an aseptic stethoscope barrier assembly in accordance with the present invention;

FIG. 2B is an exploded view of the assembly in accordance with the present invention;

FIG. 3 is a perspective view of a first step in attaching the barrier assembly to a stethoscope head;

FIG. 4A is a perspective view showing the barrier assembly attached to the stethoscope head;

FIG. 4B is a perspective view showing the removing of one of the layer of the barrier assembly to expose the aseptic barrier portion of the assembly;

FIG. 5A is a perspective view showing the discarding of one of the layers of the barrier assembly and the partial removal of the other layer of the barrier assembly;

FIG. 5B is a perspective view showing the discarding of the other layer of the barrier assembly;

FIG. 6A is a perspective view showing one packaging manner used to transport the barrier assembly;

FIG. 7A is a perspective view showing another packaging manner used to transport numerous barrier assembles; and

FIG. 7B is a perspective view showing another packaging manner used to transport numerous barrier assembles.

### DETAILED DESCRIPTION OF THE INVENTION

While the invention is susceptible to embodiments in many different forms, there are shown in the drawings and will be described herein, in detail, the preferred embodiments of the present invention. It should be understood, however, that the present disclosure is to be considered an exemplification of the principles of the invention and is not intended to limit the spirit or scope of the invention and/or the embodiments illustrated.

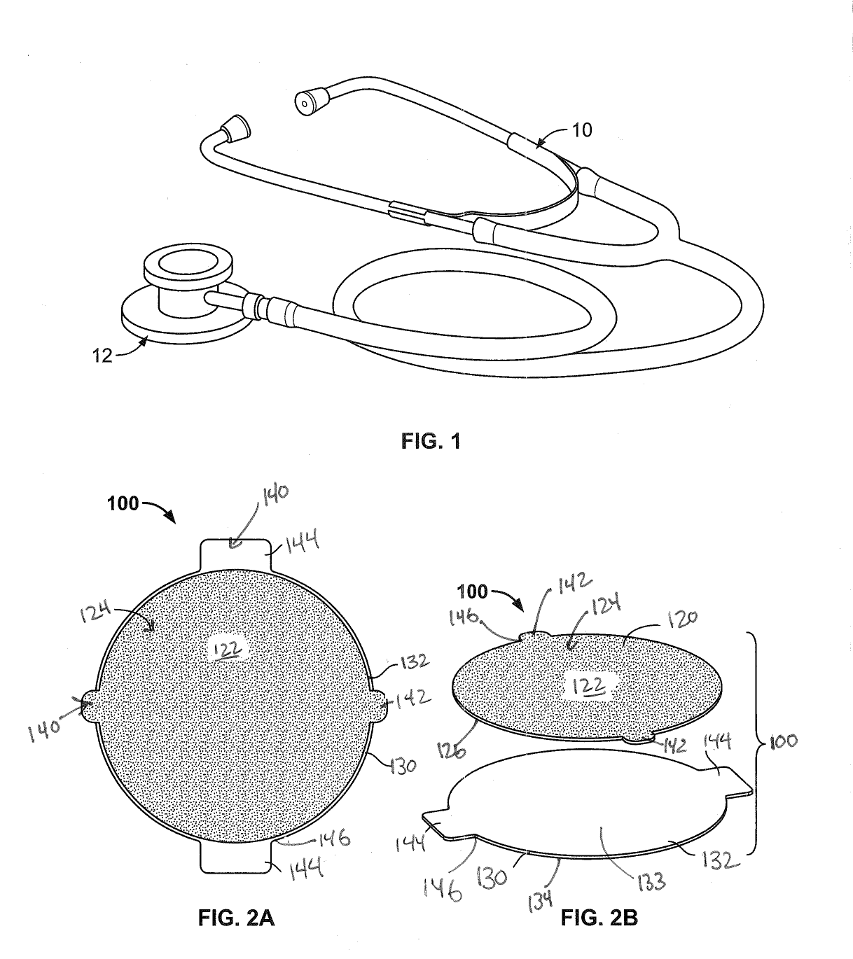

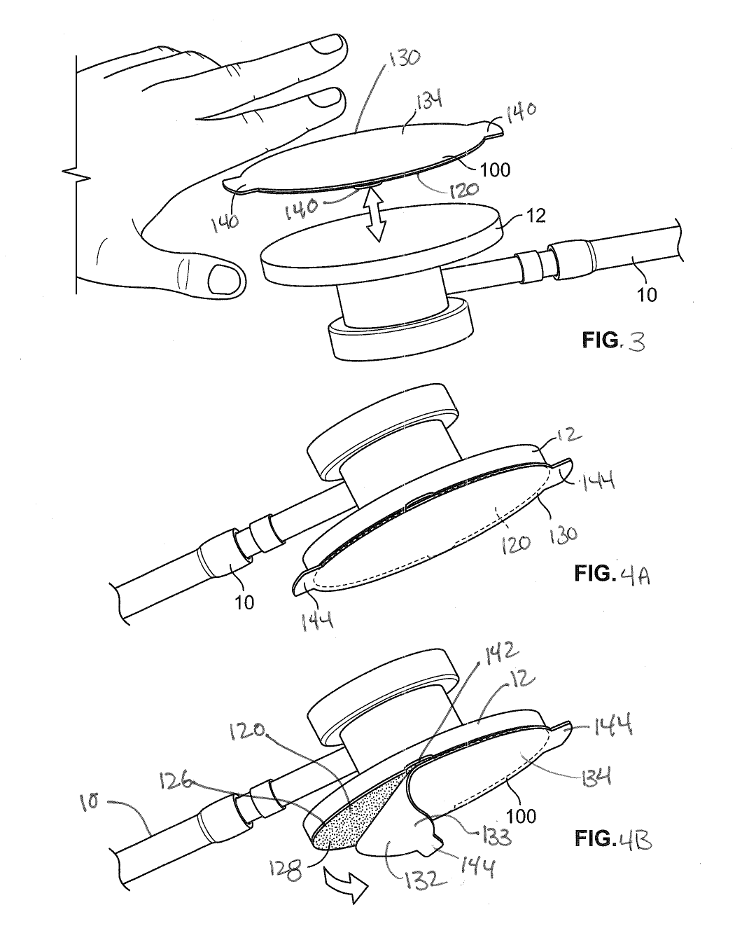

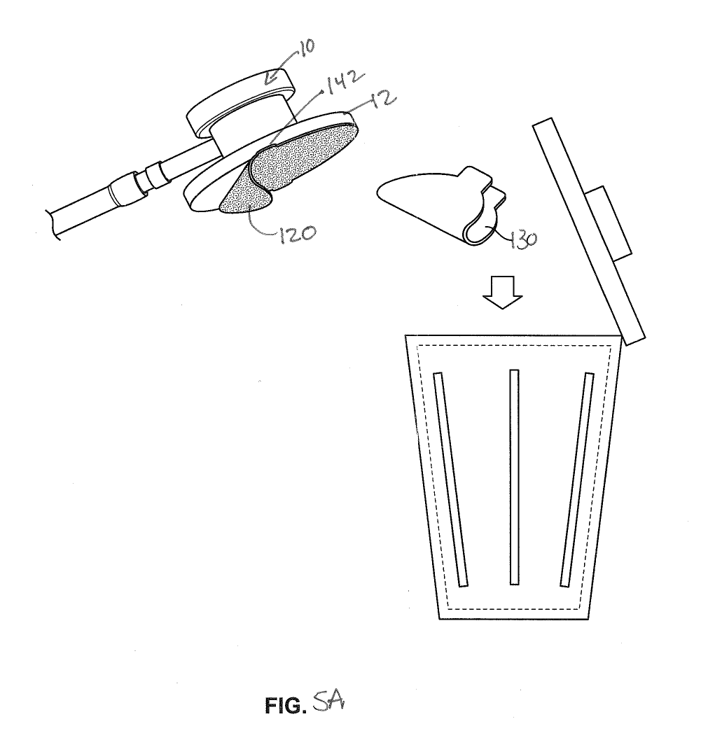

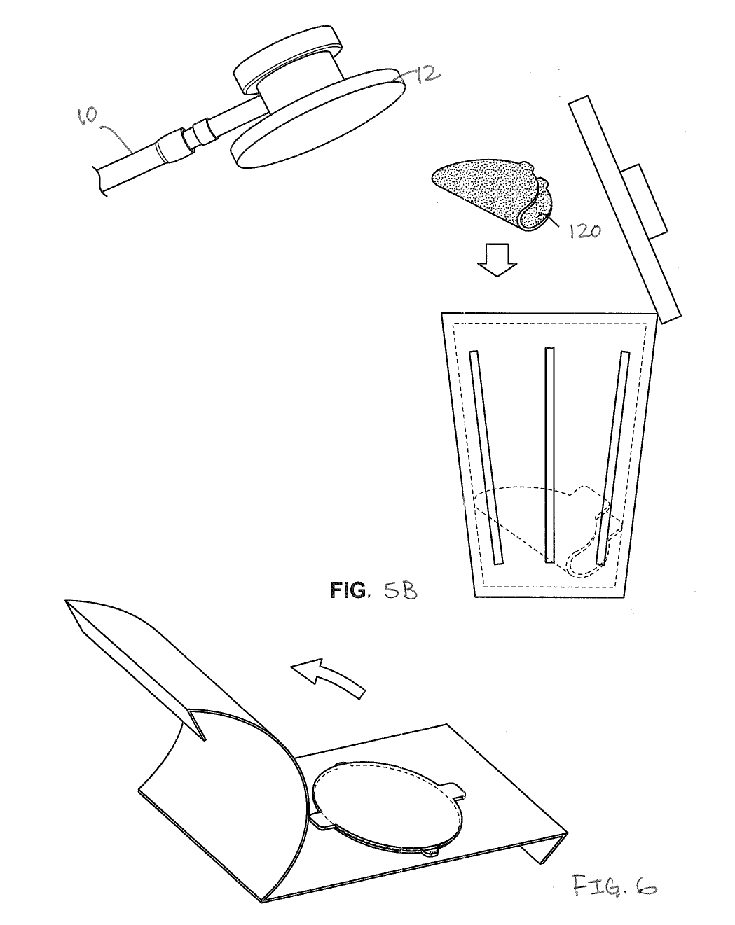

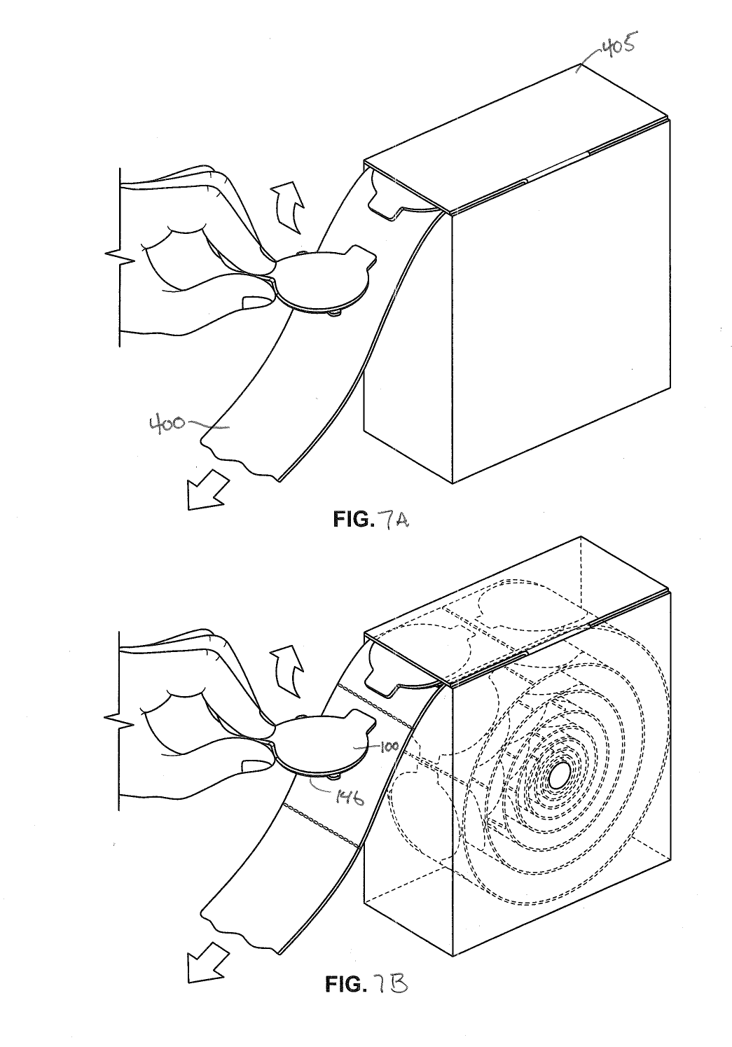

Referring now to FIG. 1, there is shown a stethoscope 10 well known in the prior art. The stethoscope generally includes a stethoscope head 12 that is placed against a patient's body to take blood pressure and to examine the chests, abdomens, and other areas of patients. The stethoscope head is comprised of a larger diaphragm and, opposite, a smaller bell. The diaphragm and bell or the stethoscope head 12 of the stethoscope 10 should be cleaned before and after use. The present invention, illustrated in FIGS. 2A and 2B, is the design of an aseptic barrier assembly 100 providing protection as a mechanical barrier between the stethoscope head 12 and the patient's skin. In other aspects one of the layers of the barrier assembly 100 may include a disinfectant or an antiseptic agent on one side to provide cleaning properties and which can be used to cover the stethoscope head 12 and provide a disinfectant or antiseptic clean side for use with a patient.

The cover assembly 100 includes two layers. A first layer 120 includes a lower most side 122 that has an adhesive material 124 that allows the first layer to be removably attached to the stethoscope head 12. The first layer 120 further includes a first middle side section 126 that is an aseptic barrier. The first middle side section 126 is overlaid with the second layer 130 of the cover assembly 100. The second layer 130 has a second middle side section 132 that faces the first middle side section 126. The middle side section 132 includes an adhesive portion 133 that allows the second layer 130 to be secured to and separable from the first layer 120. Lastly, the second layer 130 includes an upper most side 134 that a heathcare provider can touch or handle especially when applying the cover assembly to the stethoscope head 12.

In other aspects of this embodiment, the first middle side section 126 may include a disinfectant and/or an antiseptic agent, referred to herein as agent material 128. The first middle side section 126 and more importantly the agent material 128 would then be overlaid by the second layer 130 of the cover assembly 100.

Each layer 120/130 further include tab(s) 140 (first layer tabs 142 and second layer tabs 144) extending from a terminal edge 146. Multiple tabs on each layer may be provided as directed. The tabs 140 help the heathcare provider grasp the cover 100 for its use. The tabs 140 preferably extend past the edge of the stethoscope head 12, to provide the heathcare provider with an easy to grasp portion. Once the cover is secured to the stethoscope head 12 (FIG. 3), the heathcare provider can separate the first and second layers at the tabs 140 and pull away the second layer 130 at the second layer tabs 144 (FIGS. 4A and 4B) and then discard the second layer (FIG. 5A), thereby leaving the first layer 120 adhered to the stethoscope head 12 and exposing the agent material 128. After use the heathcare provider can then grasp and pull at the first layer tabs 142 to remove and discard the first layer 120 (FIG. 5B).

The barrier assembly 100 may be preferably made from a clear, latex free material and/or from a crinkle-free material to help reduce artificial noise. In addition, a generally circularly shaped cover is preferable since the stethoscope head 12 of the stethoscope 10 is circular and it is found that the circularly shaped cover would be an easy shape to grasp and use, however, other shapes may be employed if desired.

Referring now to FIGS. 6 through 7A, the barrier assemblies 100 can be packaged in various ways. Single serving covers can be individually packaged in a clear packaging 300, which opens and peels away allowing the heathcare provider to peel a cover away from the clear packaging 300 and secure immediately to a stethoscope head 12. In other packaging concepts, the barrier assemblies 100 can be affixed to a roll sheet 400 and placed in a dispenser 405 for ease of use. The method of attaching the individual barrier assemblies to the roll sheet could include a small amount of epoxy or adhesive at the tabs or around the terminal edge 146. The roll sheet 400 can further include perforations separating each barrier assembly to allow the heathcare provider to remove and discard portions of the roll sheet after removing the covers.

The dispenser 405 could further be a wall mounted device or rest on a table or counter. Either method is well known and can be employed at the convenience of the manufacturer. A pocket size variation may be employed for mobile convenience.

From the foregoing and as mentioned above, it will be observed that numerous variations and modifications may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

We claim:

1. A sterilized barrier assembly for a stethoscope defined to have at least a stethoscope head, the barrier assembly comprising:
a first layer of material having a first bottom side and a first top side, the first bottom side having an adhesive material permitting the first layer to be removably attached to the stethoscope head, the first top side having a disinfectant and/or antiseptic agent; and
a second layer of material having a second bottom side and a second top side, the second bottom side having a portion with adhesive such that the second bottom side is removably attached to the first top side,
whereby when the second layer of material is removed from the first layer of material, the disinfectant and/or antiseptic agent on the first top side is exposed to the atmosphere.

2. The sterilized barrier assembly of claim 1, wherein the first and second layers of material are generally annular sheets of flexible material having a perimeter defined to cover a stethoscope cover.

3. The sterilized barrier assembly of claim 1, further comprising:
at least one tab disposed on a terminal edge defined on each first and second layers of material.

4. A method of sterilizing a stethoscope head defined on a stethoscope, the method comprising the steps of:
(a) providing a sterilized barrier assembly defined to include:
a first layer of material having a first bottom side and a first top side, the first bottom side having an adhesive material permitting the first layer to be removably attached to the stethoscope head, the first top side having a disinfectant and/or antiseptic agent, and a second layer of material having a second bottom side and a second top side, the second bottom side having a portion with adhesive such that the second bottom side is removably attached to the first top side,
(b) applying the sterilized barrier assembly to the stethoscope head by pressing against the second top side such that the first bottom side having the adhesive material is secured to the stethoscope head;
(c) removing the second layer of material from the first layer of material exposing the disinfectant and/or antiseptic agent to the atmosphere; and
(d) removing the first layer of material after use.

5. The method of claim 4 wherein the steps of removing the first and second layer of materials include the steps of grabbing and pulling the layer of materials from at least one tab disposed on a terminal edge defined on each first and second layers of material.

6. A sterilized barrier assembly for a stethoscope defined to have at least a stethoscope head, the barrier assembly comprising:
a first layer of material having a first bottom side and a first top side, the first bottom side having an adhesive material permitting the first layer to be removably attached to the stethoscope head, the first top side having a disinfectant and/or antiseptic agent;
a second layer of material having a second bottom side and a second top side, the second bottom side having a portion with adhesive such that the second bottom side is removably attached to the first top side, whereby when the second layer of material is removed from the first layer of material, the disinfectant and/or antiseptic agent on the first top side is exposed to the atmosphere; and
a packaging medium having a first portion to secure to at least a portion of the first bottom side of the first layer of material.

7. The barrier assembly of claim 6, wherein the packaging medium is a roll of paper having a plurality of substantially identical barrier assemblies secured thereto in a spaced configuration.

8. The barrier assembly of claim 7, wherein the roll of paper include perforations between two substantially identical barrier assemblies.

9. The barrier assembly of claim 6, wherein the packaging medium includes a second portion over the second top side of the second layer of material, and wherein the second portion and the first portion each having a portion edge with adhesive to permit the second and first portions to be removably secured to each other.

10. A stethoscope head barrier assembly for a stethoscope defined to have at least a stethoscope head, the barrier assembly comprising:
a first layer of material having a first bottom side and an aseptic first top side, the first bottom side having an adhesive material permitting the first layer to be removably attached to the stethoscope head; and
a second layer of material having a second bottom side and a second top side, the second bottom side having a portion with adhesive such that the second bottom side is removably attached to the aseptic first top side,
whereby when the second layer of material is removed from the first layer of material, the aseptic first top side is exposed to the atmosphere.

11. The barrier assembly of claim 10, wherein the first and second layers of material are generally annular sheets of flexible material having a perimeter defined to cover a stethoscope cover.

12. The barrier assembly of claim 1, further comprising:
at least one tab disposed on a terminal edge defined on each first and second layers of material.

13. A barrier assembly for a stethoscope defined to have at least a stethoscope head, the barrier assembly comprising:
a first layer of material having a first bottom side and an aseptic first top side, the first bottom side having an adhesive material permitting the first layer to be removably attached to the stethoscope head;
a second layer of material having a second bottom side and a second top side, the second bottom side having a portion with adhesive such that the second bottom side is removably attached to the aseptic first top side, whereby when the second layer of material is removed from the first layer of material, the aseptic first top side is exposed to the atmosphere; and
a packaging medium having a first portion to secure to at least a portion of the first bottom side of the first layer of material.

14. The barrier assembly of claim 13, wherein the packaging medium is a roll of paper having a plurality of substantially identical barrier assemblies secured thereto in a spaced configuration.

15. The barrier assembly of claim 14, wherein the roll of paper include perforations between two substantially identical barrier assemblies.

16. The barrier assembly of claim 13, wherein the packaging medium includes a second portion over the second top side of the second layer of material, and wherein the second portion and the first portion each having a portion edge with adhesive to permit the second and first portions to be removably secured to each other.
